(19) United States
(12) Patent Application Publication
(10) Pub. No.: US 2009/0264859 A1
(43) Pub. Date: Oct. 22, 2009

(54) Catheter Having a Selectively Expandable Distal Tip

(75) Inventor: Juan-Pablo Mas, Indianapolis, IN (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/326,591

(22) Filed: Dec. 2, 2008

Related U.S. Application Data
(60) Provisional application No. 61/046,492, filed on Apr. 21, 2008.

(51) Int. Cl. A61M 25/10; A61F 2/84

(52) U.S. Cl. 604/509; 604/103.05; 604/96.01; 623/1.11

(57) ABSTRACT

A catheter for use in a medical procedure includes an elongate outer member, an elongate inner member and an expandable distal tip attached to a distal most end of at least one of the outer member or inner member, wherein the distal tip has a first shape and a second shape and the tip can be easily transformed between the first and second shapes to aid in navigation through a patient's vascular system. A method of using the catheter includes inserting the catheter into the vascular system, navigating the distal end of the catheter through the vascular system, determining an obstruction within the vascular system, modifying the expandable tip from the first shape to the second shape in response to the determination of the obstruction, advancing the modified expandable tip passed the determined obstruction and returning the modified expandable tip to the first shape upon passing the determined obstruction.

### RELATED APPLICATIONS

This application claims priority to and the benefit of U.S. Provisional Application No. 61/046,492, filed Apr. 21, 2008, entitled “Catheter having a Selectively Expandable Distal Tip” to Juan-Pablo Mas, the entirety of which is incorporated herein by reference.

### FIELD OF INVENTION

This invention relates generally to medical devices and procedures, and more particularly to a device and system for delivering an implantable medical device to a location in a vascular system.

### BACKGROUND OF THE INVENTION

Heart valves, such as the mitral, tricuspid, aortic and pulmonary valves, are sometimes damaged by disease or by aging, resulting in problems with the proper functioning of the valve. Heart valve problems generally take one of two forms: stenosis, in which a valve does not open completely or the opening is too small, resulting in restricted blood flow; or insufficiency, in which blood leaks backward across a valve when it should be closed.

Previously, valve repair or replacement required open-heart surgery with its attendant risks, expense, and extended recovery time. Open-heart surgery also requires cardiopulmonary bypass with risk of thrombosis, stroke, and infarction. More recently, flexible valve prostheses and various delivery devices have been developed so that replacement valves can be implanted transvenously using minimally invasive techniques.

Recently, implantable heart valves have been developed that can be delivered transvenously using a catheter-based delivery system. These valves comprise a collapsible valve attached to the interior of a tubular frame or stent. The valve can be any of the valve prostheses described above, or it can be any other suitable valve. In the case of valves in harvested vessels, the vessel can be of sufficient length to extend beyond both sides of the valve such that it extends to both ends of the valve support stent.

The valves can also comprise a tubular portion or “stent graft” that can be attached to the interior or exterior of the stent to provide a generally tubular internal passage for the flow of blood when the leaflets are open. The graft can be separate from the valve and it can be made from any suitable biocompatible material including, but not limited to, fabric, a homograft, porcine vessels, bovine vessels, and equine vessels.

The stent portion of the device can be reduced in diameter, mounted on a catheter, and advanced through the circulatory system of the patient. The stent portion can be either self-expanding or balloon expandable. In either case, the stented valve can be positioned at a delivery site, where the stent portion is expanded against the wall of a previously implanted prosthesis, or against the wall of a native vessel or heart chamber to hold the valve firmly in place.

During delivery of these valves, the catheter is maneuvered, until the end of the catheter is positioned in the vicinity of the intended treatment site. Generally, an inner tube of a delivery catheter is held stationary while the sheath of the delivery catheter is withdrawn. For a self expanding configuration the inner tube prevents the stent-graft from moving back as the sheath is withdrawn.

As the sheath is withdrawn, the stent is gradually exposed from a proximal end to a distal end of the stent-graft, the exposed portion of the stent-graft radially expands so that at least a portion of the expanded portion is in substantially conforming surface contact with a portion of the interior of the lumen (e.g., arterial wall).

In straight anatomies, delivery of an implantable device by catheter is relatively straightforward. However, delivery can be difficult in complex anatomies. Examples of such difficult procedures are catheter delivery of a prosthetic aortic valve, catheter delivery of a prosthetic pulmonic valve, and catheter delivery of a prosthetic mitral valve; all of which present a significantly complex route for navigation by a delivery catheter with a relatively large diameter.

Thus, it would be desirable to provide devices and systems that will allow navigation through difficult, tortuous, and complex anatomy by relatively large diameter catheters for delivery of implantable devices. It would also be desirable to provide method for using such devices and systems.

### SUMMARY OF THE INVENTION

The present invention discloses a selectively expandable tip section for catheters that can be used for assisting in navigation through complex vascular anatomy. As such, one aspect of the invention provides a catheter for use in a medical procedure comprising an elongate, flexible, generally tubular outer member; an elongate, flexible generally tubular inner member, the inner and outer members each having a proximal section, a distal section, and a central lumen passing therethrough; and an expandable distal tip attached to the distal most end of at least one of the outer member or inner member, wherein the distal tip has a first shape and a second shape and the tip can be easily transformed between the first and second shapes to aid in navigation through a patient's vascular system.

Another aspect of the present invention provides a method for navigating a catheter through a patient's vascular system. The method includes inserting a catheter into the vascular system. The catheter for use in this method comprises an elongate, flexible, generally tubular outer member; an elongate, flexible generally tubular inner member, the inner and outer members each having a proximal section, a distal section, and a central lumen passing therethrough; and an expandable distal tip attached to the distal most end of at least one of the outer member or inner member, wherein the distal tip has a first shape and a second shape and the tip can be transformed between the first and second shapes to aid in navigation through a patient's vascular system. The method further includes navigating the distal end of the catheter through the vascular system, determining an obstruction within the vascular system, and modifying the expandable tip from the first shape to the second shape in response to the determination of the obstruction. The method may further include advancing the modified expandable tip passed the determined obstruction; and returning the modified expandable tip to the first shape upon passing the determined obstruction.

The aforementioned and other features and advantages of the invention will become further apparent from the following detailed description of the presently preferred embodiments, read in conjunction with the accompanying drawings, which are not to scale. The drawings should not be taken to limit the invention to the specific embodiments, but are for explanation and understanding. The detailed description and drawings are merely illustrative of the invention rather than limiting, the scope of the invention being defined by the appended claims and equivalents thereof.

### BRIEF DESCRIPTION OF THE DRAWINGS

FIG. 1 is a schematic interior view of heart showing the interior structure of the heart;

FIG. 2 is a plan view of a heart showing the location of the heart valves;

FIGS. 3A and 3B show one embodiment of a catheter according to the current invention;

FIGS. 4A and 4B show another embodiment of a catheter according to the current invention;

FIG. 5 is a schematic view of a heart showing the positioning of a catheter in the right ventricle and the puncturing of the septum according to the current invention;

FIG. 6 is a schematic view of a heart showing a catheter of the prior art in a right ventricle and a catheter with a tip according to the current invention in the left ventricle;

FIGS. 7A and 7B are schematic views of a heart showing the positioning of a catheter with an expandable tip in the aortic arch according to the current invention;

FIGS. 8, 9A, 9B, 10A and 10B show alternate embodiments of catheter tips according to the current invention; and

FIG. 11 is a flow chart of one embodiment of a method of using a catheter having an expandable tip according to the current invention.

### DETAILED DESCRIPTION

The invention will now be described by reference to the figures wherein like numbers refer to like structures. The terms “distal” and “proximal” are used herein with reference to the treating clinician during the use of the catheter system; “Distal” indicates an apparatus portion distant from, or a direction away from the clinician and “proximal” indicates an apparatus portion near to, or a direction towards the clinician.

The current invention discloses devices having an expandable or reshapable tip useful for traversing the vascular system of a patient. The use of this expandable tip would benefit clinicians trying to track a delivery system through the chambers of the heart for structural heart repair, valvuloplasty, ICD/pacemaker lead delivery, etc. The usage of this system would be very similar to traditional replacement valve delivery systems, in that it rides over a guidewire and has an outer sheath to protect the device. The current invention would provide a clinician with an advantage when trying to navigate a delivery system past anatomical features, such as, for example valve annulus, leaflets, chordae, aortic arch and the like. A reshaping delivery system would serve to assist these sometimes challenging situations of tracking in the heart. For example, when the distal tip needs to be large or bulbous to avoid getting tangled in between the chordae. Also, when the most distal tip of the system is caught on an anatomical feature (e.g. the aortic valve annulus on a retrograde approach, etc.), partial reshaping of the tip allows for minor course correction that can be just sufficient to bypass the feature or redirect the tip.

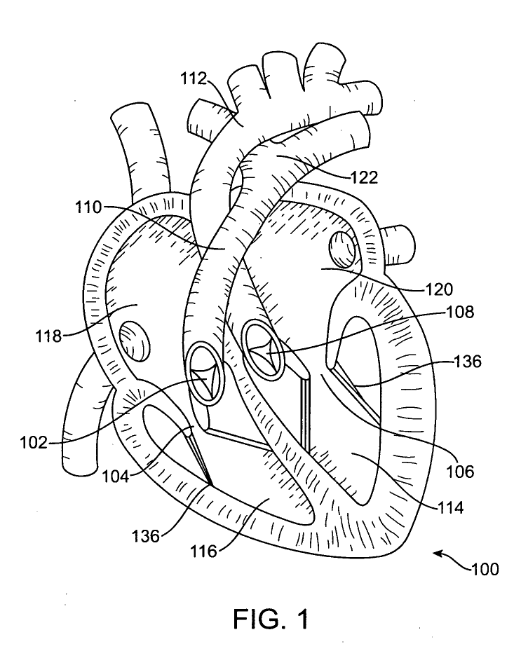

Referring to the drawings, FIG. 1 is a schematic representation of the interior of human heart 100. Human heart 100 includes four valves that work in synchrony to control the flow of blood through the heart. Tricuspid valve 104, situated between right atrium 118 and right ventricle 116, and mitral valve 106, between left atrium 120 and left ventricle 114 facilitate filling of ventricles 116 and 114 on the right and left sides, respectively, of heart 100. Also shown in the figure are chordae tendenae 136, attached to the valve leaflets and papillary muscle.

Aortic valve 108 is situated at the junction between aorta 112 and left ventricle 114 and facilitates blood flow from heart 100, through aorta 112 to the peripheral circulation. Pulmonary valve 102 is situated at the junction of right ventricle 116 and pulmonary artery 110 and facilitates blood flow from heart 100 through the pulmonary artery 110 to the lungs for oxygenation. The four valves work by opening and closing in harmony with each other.

During diastole, tricuspid valve 104 and mitral valve 106 open and allow blood flow into ventricles 114 and 116, and the pulmonic valve and aortic valve are closed. During systole, shown in FIG. 1, aortic valve 108 and pulmonary valve 102 open and allow blood flow from left ventricle 114, and right ventricle 116 into aorta 112 and pulmonary 110, respectively.

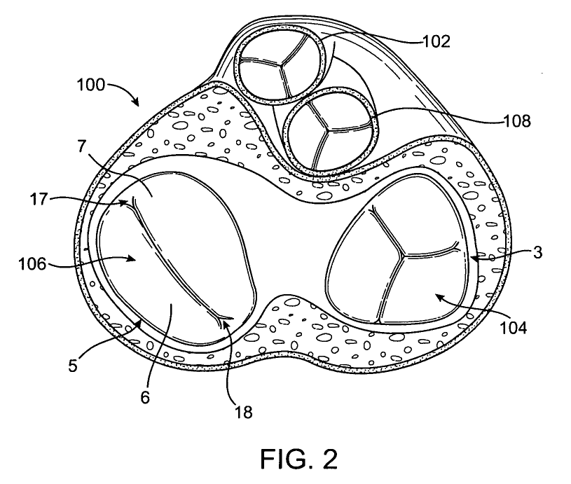

FIG. 2 shows a plan view of a cross-section of heart 100 having tricuspid valve 104 and tricuspid valve annulus 3. Mitral valve 106 is adjacent mitral valve annulus 5. Mitral valve 106 is a bicuspid valve having anterior cusp 7 and posterior cusp 6. Anterior cusp 7 and posterior cusp 6 are often referred to, respectively, as the anterior and posterior leaflets. Also shown in the figure are the posterior commisure 17 and the anterior commisure 18.

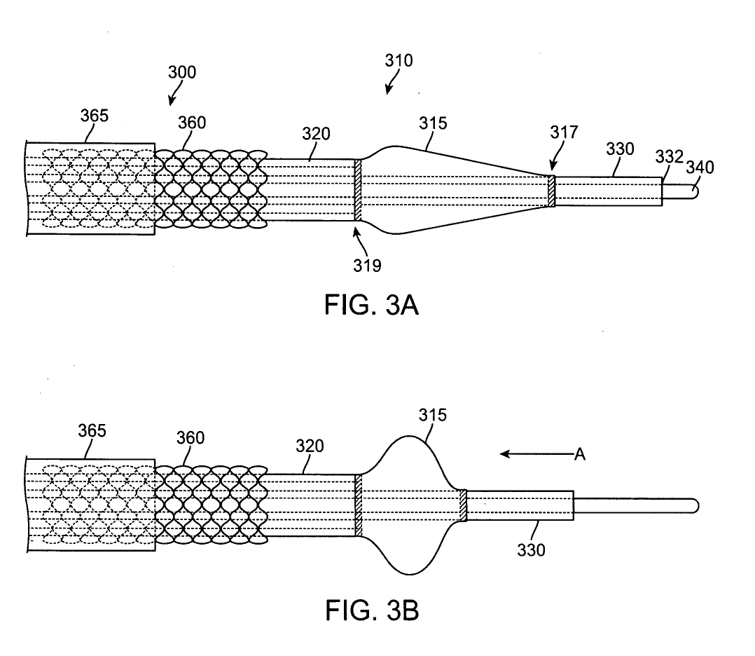

Referring to FIG. 3A and FIG. 3B, there can be seen a catheter 300 having a distal end 310 with a selectively expandable distal tip 315. FIG. 3A illustrates expandable tip 315 in a delivery configuration having a first shape and FIG. 3B illustrates expandable tip 315 in an expanded configuration having a second shape. The catheter in the depicted embodiment is a concentric system having an elongated outer member 320 and an elongated inner member 330. At least one lumen 332 communicates through the length of inner member 330 and a guidewire 340 is extended through the lumen. In FIG. 3A, the expandable distal tip 315 of catheter 300 is in a cone shaped delivery configuration while FIG. 3B shows the expandable distal tip 315 in its expanded state. In one embodiment, the relative distance between the inner and outer members is used to control the size and shape of the expandable portion of the expandable tip. In an example, the shape of distal tip 315 shown in FIG. 3B is a generally bulbous shape. In use, the clinician may desire a less bulbous (i.e. more conical) inflated shape which may be obtained by keeping a greater relative distance between the inner member and the outer member as compared to the relative distance required to obtain the bulbous form. The cone shape of the expandable tip helps the distal end of the catheter to track easily through the vasculature and the expanded configuration of tip 315 (shown in FIG. 3B) allows the tip to be used for tracking through obstacles in the vasculature. The first shape of the expandable tip for the delivery configuration may take other forms such as a parabolic shape.

Delivery systems with the expandable tip of the current invention will track through a variety of anatomy on its course to its desired end point, and thus a device with an adaptable, reshapable tip is beneficial. In one embodiment, expandable tip 315 is an expandable braided core that is constructed in a manner similar to other braided vascular devices, such as filters. Examples of braided devices can be found in U.S. Pat. No. 7,044,958, to Douk et al. and U.S. Pat. No. 6,716,231, to Rafiee et al., the contents of both are incorporated herein by reference thereto.

In one embodiment, a braided under portion, or layer, of expandable tip 315 is covered with an elastomeric, hydrophilic material that allows it to change shape from a low-profile shape, to a bulbous and otherwise atraumatic shape when necessary to traverse around or through various anatomical features. For instance the chordae, tribeculae, and leaflets could benefit from an atraumatic device tip during tracking in the heart. In one embodiment, the invention comprises a delivery system with adaptable tip feature whose shape depend on the anatomy through which the tip is traversing.

Expandable tip 315 can be constructed by braiding super-elastic wire (e.g. Nitinol, MP35N, etc.) into a tubular shape and then heat-setting the braid into the conical shape of expandable tip 315. In another embodiment, the braided layer of expandable tip 315 is disposed over a plurality of struts. The struts may be made of a metallic or polymeric material, or a combination thereof. The struts may provide support for the braided layer as well as strength and flexibility during navigation of the vasculature.

This heat-set braid would next be coated in an elastomeric sealant, such as, for example, silastic, silicone, urethane, and the like. The elastomeric sealant can be lubricious or coated with a hydrophilic material. As an alternative to the coating with an elastomeric sealant, a thin over-molding of the braided section with a soft-durometer material, such as, for example and without limitation, PEBAX or other elastic nylon, provides a thin walled structure that is able to deform.

In one embodiment, this formed tip is mounted and bonded onto a concentric two catheter system such as catheter 300 illustrated in FIGS. 3A and 3B. In this embodiment, a distal end 317 of expandable tip 315 is bonded to inner member 330 and a proximal end 319 of expandable tip 315 bonded to outer member 320. The expandable tip is bonded and sealed to the respective catheters by, for example, an adhesive, heat shrinking, or a polymer fusion process. In this embodiment, the position of these two concentric catheter members 320, 330 relative to each other is moveable, thereby causing the expansion and collapse of braided expandable tip 315. For example, FIG. 3B shows the expansion of tip 315 whereby outer member 320 is held relatively stationary and inner member 330 is pulled in a proximal direction, represented by arrow A. In this embodiment, movement of inner member 330 in a direction opposite of arrow A, will return expandable tip 315 to the position illustrated in FIG. 3A. In another embodiment, movement of expandable tip 315 is affected by movement of outer member 320 relative to a stationary inner member 330. In yet another embodiment, movement of expandable tip 315 is affected by the movement of both outer member 320 and inner member 330 relative to each other. In one embodiment, the relative distance between the inner member and the outer member controls the size and shape of the expandable tip 315.

The shape of the expandable tip is controllable by the user at the device handle. The tip can be used on catheters that have other means for orienting the tip of the catheter or they can be placed on catheters that have no direction controls. An example of a catheter with means for steering the catheter can be found in the U.S. Patent App. with the publication No. 2007/0225681, and the contents of that application are incorporated herein by reference.

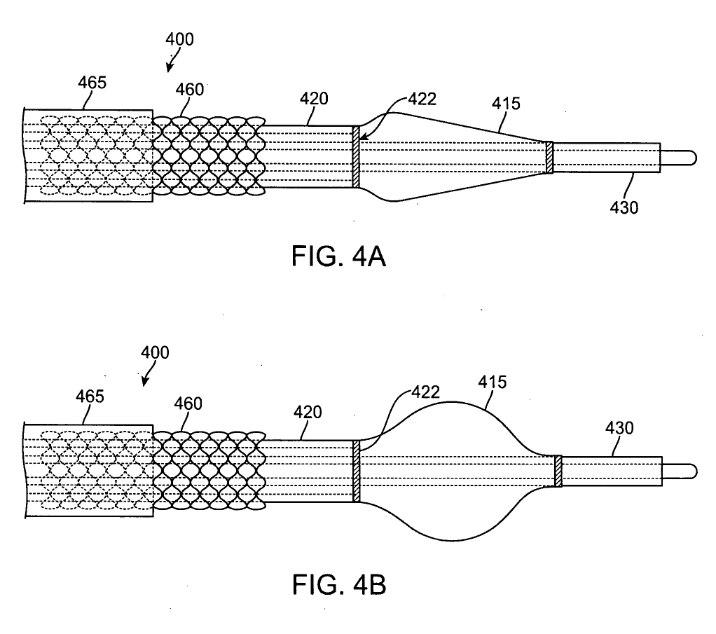

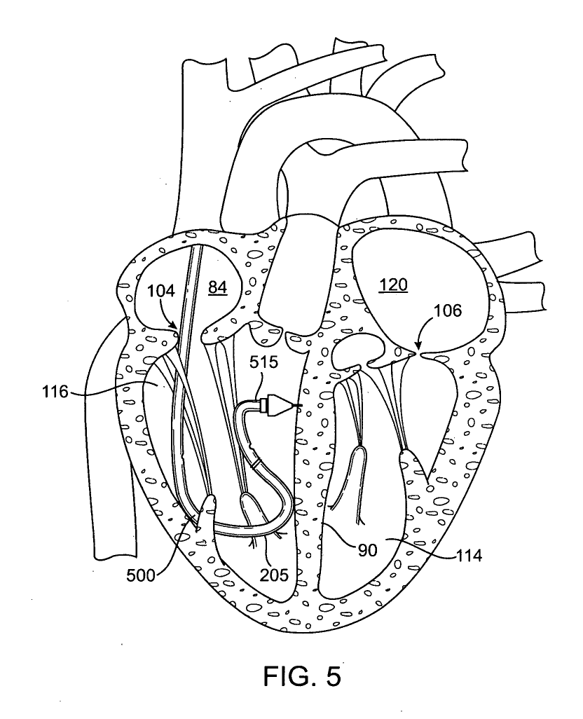

FIGS. 4A and 4B illustrate another embodiment of a catheter 400 having a ballooning version of the expandable tip according to the present invention. FIG. 4A illustrates expandable tip 415 in a delivery configuration having a first shape and FIG. 4B illustrates expandable tip 415 in an expanded configuration having a second shape. The expandable tip 415 is made in a similar fashion as that of expandable tip 315. However, in this embodiment, the deformation of expandable tip 415 does not require the relative motion of a two catheter system as described above. In this embodiment, saline or radiopaque dye pressurization via inflation lumen 422 would allow the user to change the shape/state of the expandable tip 415. Expandable tip 415 would also benefit from having a braided core for structural support. In one embodiment, a braided core is composed to return the expandable tip to a pre-defined shape/size upon deflation of the tip. An inflation lumen 422, defined by the space between the OD of inner member 430 and the ID of the outer member 420, would be used to infuse and inflate the balloon-like tip. In one embodiment, the relative distance between the inner member 430 and the outer member 420 controls the size and shape of the expandable tip 415.

FIGS. 3A to 4B illustrate embodiments of the present invention that include an expandable medical device 360, 460, for example, a stent or a stent mounted heart valve, disposed on the system near the distal end. In at least one embodiment, the system includes a delivery sheath 365, 465 disposed over an expandable medical device. 360, 460. In at least one embodiment, the expandable device is self expanding, such as a self-expanding stent. In another embodiment, the outer member is a balloon catheter which may or may not include a balloon expandable medical device disposed over the balloon.

The catheters, elongated members and sheaths of the invention can be made from flexible, biocompatible polymeric materials that are suitable for catheter construction. Examples of such material include, but are not limited to, polyurethane, polyethylene, nylon and polytetrafluoroethylene (PTFE). At least one embodiment of the invention can include a reinforced layer of biocompatible material for at least one of the inner member or outer member. The material can be any material known by those having ordinary skill in the art to be suitable for constructing catheters, including PEBAX.

Embodiments of the devices disclosed or discussed herein can include materials having a high X-Ray attenuation coefficient (radiopaque material) such that the procedure may be visualized. The material can be placed or located on the devices in a manner that would be readily apparent to one of ordinary skill in the art. In one embodiment of the current invention, the catheter and the distal tip each have bands of radiopaque material spaced along a portion thereof. Examples of suitable radiopaque material include, but are not limited to, gold, tungsten, silver, iridium, platinum, barium sulfate and bismuth sub-carbonate. The procedure may be visualized using fluoroscopy, echocardiography, intravascular ultrasound, angioscopy, or other means of visualization.

FIG. 5 shows a catheter 500 having an expandable distal tip 515 according to the current invention. Catheter 500 may be the same as, or similar to, catheter 300, 400 and tip 515 may be the same as, or similar to, expandable tips 315, 415, described above. Catheter 500 is passed through the right atrium 118 and the tricuspid valve 104. The catheter has been manipulated to form a curve 205 at the distal end of the catheter such that the catheter is braced against the walls of the right ventricle 116, such that it rests against the septum 90 and the opposite free wall of the heart chamber.

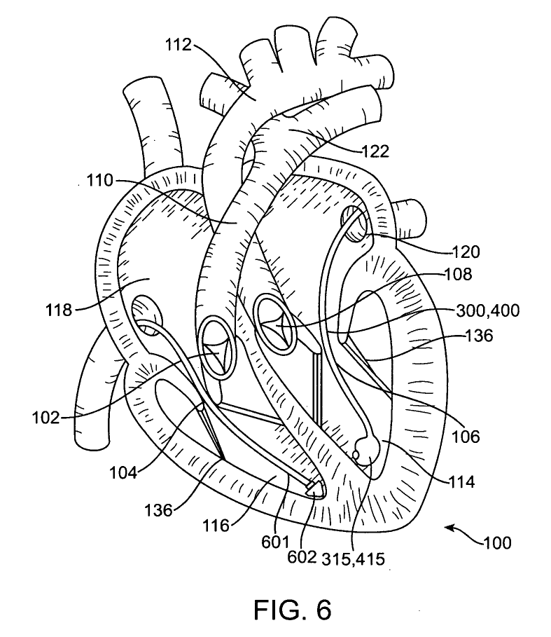

During various medical procedures it can be common for a catheter to temporarily become stuck or engaged in an obstacle or structure in the vasculature or heart. Examples of such structure or obstacles can include chordae tendinae, valve leaflets, trabecula inside of a heart chamber, a ventricular apex, etc. FIG. 6 shows a standard catheter 601 having a conical distal tip 602 that is wedged into and temporarily stuck in the apex of a right ventricle of a heart 100. In some cases, a clinician may have to exert enough force, or rotate the catheter in such a way, when attempting to dislodge the tip, that it causes injury to the heart wall, valve leaflets, chordae, or other structures. The catheter in the left ventricle of FIG. 6 is a catheter 300, 400 with an expandable tip such as expandable tip 315, 415 according to the current invention. As shown, expansion of expandable tip 315, 415 can dislodge and/or redirect the distal end of catheter 300, 400 so that the distal end does not get stuck in the apex of the left ventricle.

To use catheters having selectively expandable tips according to the current invention, a clinician can initially navigate through the vasculature in a manner similar to current catheters. When a clinician encounters some resistance to navigation, or when an obstacle is visualized using standard visualization techniques, the tip can be expanded to either dislodge the catheter from an obstacle or to keep the catheter from becoming engaged in an obstacle. By expanding the tip, a clinician may reduce the chance that there will be an injury to valve leaflets, chordae, heart walls and vessel walls. Once the catheter had been dislodged or the tip has bypassed the obstacle, the tip can be collapsed back to the conical shape for streamlined navigation through the vasculature.

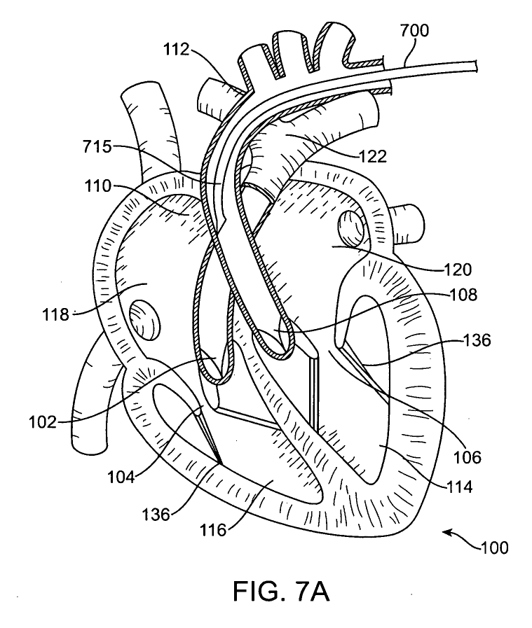

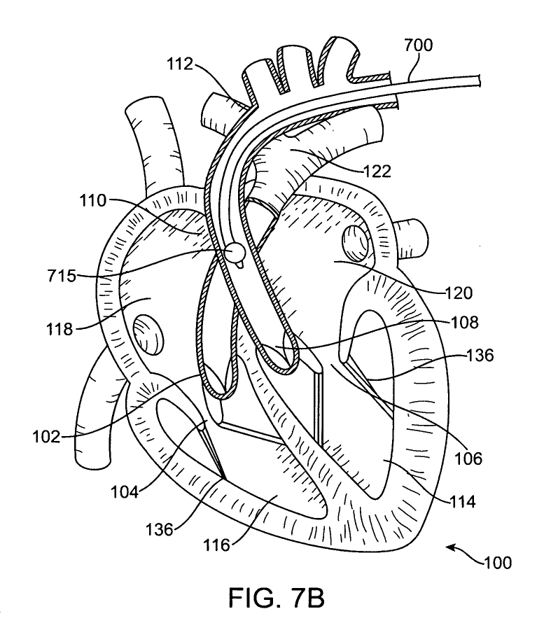

FIGS. 7A and 7B show a catheter 700 with an expandable tip 715, according to the present invention, traversing the aorta 112 of heart 100, illustrated in FIG. 1. During advancement of a catheter through the vasculature, the distal end of the catheter often abut vascular walls causing the clinician to exert force on the catheter to dislodge or otherwise maneuver the tip so that the clinician can continue to advance the catheter tip to the treatment site. One such difficult passage within the body is the aortic arch. Prior art devices without sufficient flexibility have trouble navigating the bend in the aortic arch, often getting the tip stuck at the aortic root. FIG. 7A illustrates the distal end of catheter 700 abutting the wall of the aortic arch. In this situation, a clinician using catheter 700 having an expandable tip 715 expands, mechanically or by inflation, the tip as shown in FIG. 7B to redirect the tip and to point the tip towards the aortic valve 108. Once redirected, the clinician compresses or deflates expandable tip 715 and then can continue advancing the distal end of catheter 700 towards and/or through aortic valve 108 as needed.

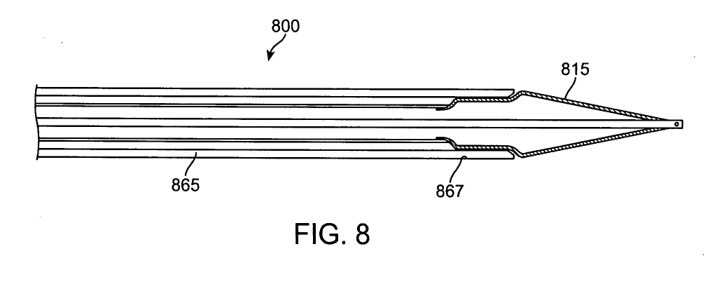

FIG. 8 shows a catheter 800 having a selectively expandable distal tip 815 according to the current invention. Catheter 800 includes sheath 865. In the embodiment depicted, the distal end 867 of sheath 865 is flush with expandable distal tip 815. This allows for additional ease for navigation through the vasculature as well as for navigation past obstacles in the vasculature.

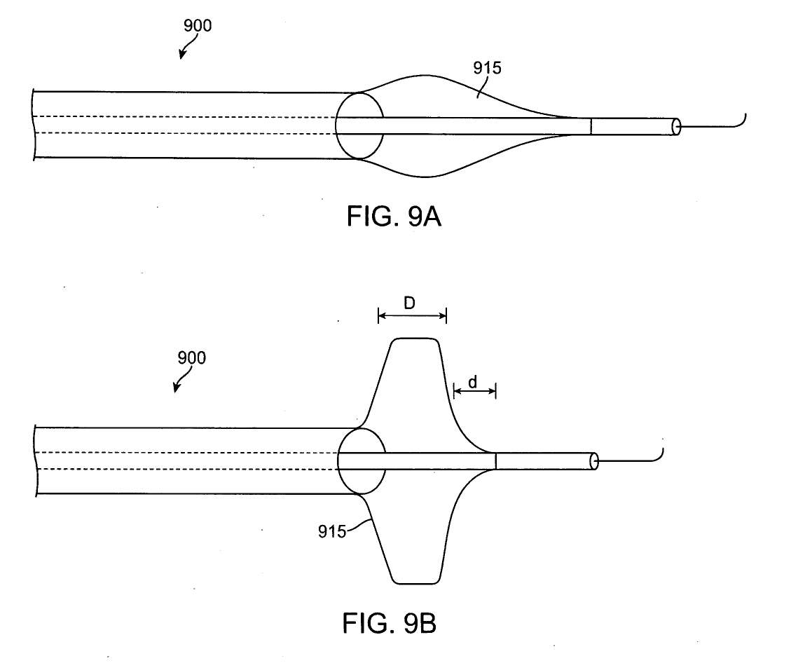

FIGS. 9A and 9B show another embodiment of a catheter 900 with a selectively expandable distal tip 915 according to the current invention. FIG. 9A shows an expandable tip on a catheter having a conical delivery system tip shape. FIG. 9B shows an expanded shape that has a length of the tip D that opens wide, and a length of the tip d that does not open, or minimally opens. This embodiment may be useful for course correction of the distal end of the delivery system tip because it acts as a means of articulation while allowing a low profile distal pointed section that can still engage an ostium or an orifice when advanced. The relative values of d and D can be modified to optimize the system for different applications.

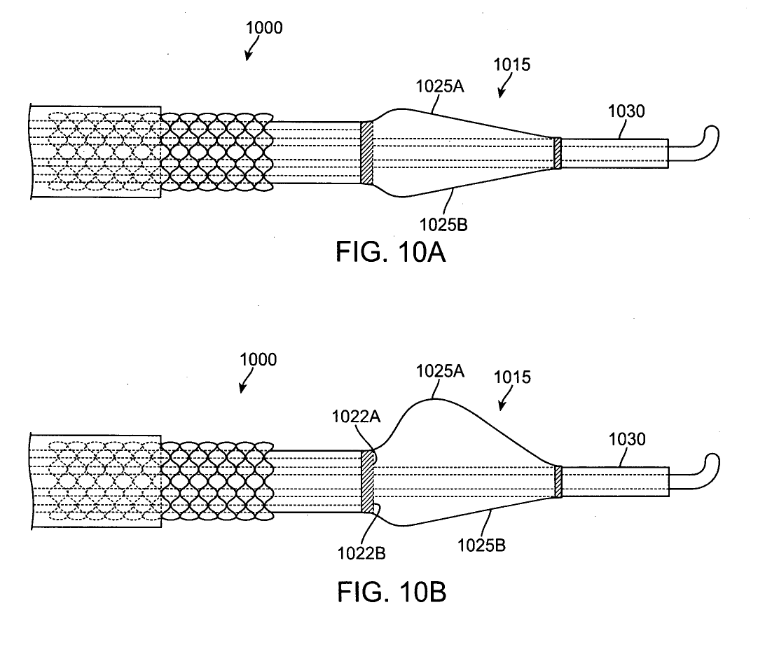

FIGS. 10A and 10B show another embodiment of a catheter 1000 with a selectively expandable distal tip 1015 according to the current invention. In this embodiment, expandable distal tip 1015 includes separate expansion chambers 1025A and 1025B. In one embodiment, expandable distal tip 1015 is a balloon having an internal longitudinal divider separating balloon 1015 into expansion chambers 1025A and 1025B. In another embodiment, balloon 1015 is bonded to inner member 1030 along opposite sides of the longitudinal portion of inner member 1030 that is positioned within balloon 1015 creating a lobed balloon when at least a portion of the balloon is inflated. Catheter 1000 includes at least two inflation lumens 1022A, 1022B for selectively inflating expansion chambers 1025a and 1025B. In use, in one embodiment, inflation of one expansion chamber 1025 directs the distal end of catheter 1000 in a direction opposite of the inflated portion, when the expandable portion abuts an anatomical feature to effectively steer the tip in a desired direction or to dislodge the tip from an obstruction. Expandable tip 1015 may be divided into two or more expandable portions. In one embodiment, expandable tip 1015 includes three expansion chambers 1025. In another embodiment, expandable tip 1015 includes four expansion chambers 1025.

The selectively expandable distal tips disclosed herein are advantageous over previously disclosed devices in that they provide a firm stable support for bracing a catheter in a heart chamber while a prosthetic heart valve is being implanted. The catheters disclosed herein also allow a clinician to change the shape of the tip to help orient the catheter when the clinician desires that the catheter be advanced around an obstacle or curve in the vasculature. The tips disclosed herein can also be used to prevent catheters and devices from damaging or becoming entangled with heart valve leaflets, chordae, or other vascular and cardiac structures.

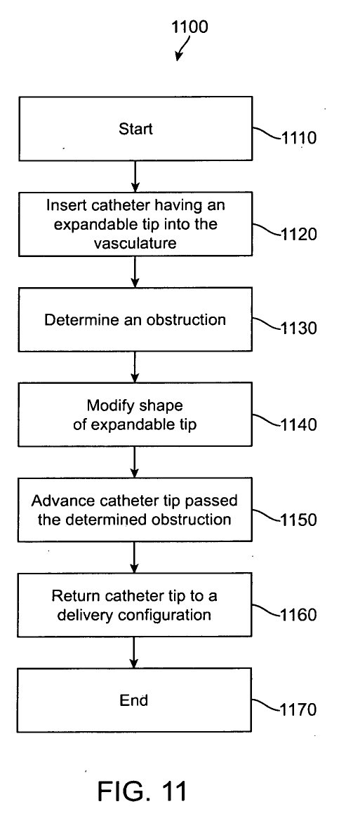

FIG. 11 is a flowchart illustrating one embodiment of a method 1100 for navigating a catheter through a patient's vascular system according to the current invention. Method 1100 may utilize any of the catheters and expandable tips described above and illustrated in FIGS. 3A to 10B, in accordance with the present invention. Method 1100 begins at Block 1110. To practice the current invention, a clinician inserts a catheter having an expandable tip into the patient's vasculature (Block 1120). At Block 1130, the clinician determines an obstruction. The determination may be made by visualization using any technique as known to those with ordinary skill in the art or by a perceived stoppage of the advancement of the catheter. The obstruction may be any of those described above. Alternatively, the clinician may determine a desired change of direction so as to navigate into a particular vessel or around a bend or sharp turn within the vessel. Based on this determination, the expandable tip is modified from a first shape delivery configuration to a second shape expanded configuration (Block 1140). The expandable tip may be modified by inflation or mechanically by movement of one or both of an inner or outer member to which the expandable tip is attached, as described above. Once the desired degree of expansion has been achieved, the expanded tip is advanced passed the obstruction (Block 1150). The expandable tip is returned to a delivery configuration upon a determination that the obstruction has been avoided or passed through and navigation of the catheter tip can continue to the treatment site (Block 1160). Any additional obstructions or changes in direction may be treated in the same or similar fashion as required to reach the treatment site. Method 1100 ends at Block 1170.

The current application describes selectively expandable distal tips for catheters used in a patient's vascular system. While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A catheter for use in a medical procedure comprising:
an elongate, flexible, generally tubular outer member;
an elongate, flexible generally tubular inner member;
the inner and outer members each having a proximal section, a distal section, and a central lumen passing therethrough; and
an expandable distal tip attached to the distal most end of at least one of the outer member or inner member, wherein the distal tip has a first shape and a second shape and the tip can be easily transformed between the first and second shapes to aid in navigation through a patient's vascular system.

2. The catheter of claim 1 wherein the expandable distal tip is constructed from a braided mesh covered with a flexible, elastomeric material.

3. The catheter of claim 1 wherein a proximal end of the expandable distal tip is attached to an outer surface of the outer member and a distal end of the expandable distal tip is attached to an outer surface of the inner member.

4. The catheter of claim 3 further comprising at least one inflation lumen disposed between the outer surface of the inner member and an inner surface of the outer member, the inflation lumen in fluid communication with an inner surface of the expandable distal tip.

5. The catheter of claim 2 wherein the distal tip includes struts inside of the braided mesh construction.

6. The catheter of claim 1 wherein the expandable distal tip is an expandable balloon.

7. The catheter of claim 6 wherein the expandable balloon comprises at least two separately inflatable expansion chambers.

8. The catheter of claim 1 where in the first shape of the distal tip is one of a conical shape or a parabolic shape.

9. The catheter of claim 1 wherein the second shape of the distal tip is bulbous.

10. The catheter of claim 1 further comprising an expandable medical device disposed on an outer surface of a distal end of the outer member of the catheter.

11. The catheter of claim 10 further comprising a sheath disposed over the expandable medical device, wherein the expandable medical device comprises a self-expanding stent.

12. The catheter of claim 11 wherein the sheath further comprises a tapered distal end.

13. The catheter of claim 10 wherein the expandable medical device comprises a stented valve.

14. A method for navigating a catheter through a patient's vascular system comprising:
inserting a catheter into the vascular system, the catheter comprising:
an elongate, flexible, generally tubular outer member;
an elongate, flexible generally tubular inner member, the inner and outer members each having a proximal section, a distal section, and a central lumen passing therethrough; and
an expandable distal tip attached to the distal most end of at least one of the outer member or inner member, wherein the distal tip has a first shape and a second shape and the tip can be transformed between the first and second shapes to aid in navigation through a patient's vascular system;
navigating the distal end of the catheter through the vascular system;
determining an obstruction within the vascular system;
modifying the expandable tip from the first shape to the second shape in response to the determination of the obstruction;
advancing the modified expandable tip passed the determined obstruction; and
returning the modified expandable tip to the first shape upon passing the determined obstruction.

15. The method of claim 14 wherein modifying the expandable tip from the first shape to the second shape comprises inflating the expandable tip.

16. The method of claim 14 wherein the expandable tip further comprises at least two separately inflatable expansion chambers.

17. The method of claim 14 further comprising:
navigating the expandable distal tip to a treatment site; and
deploying a medical device at the treatment site.
